(19) United States
(12) Patent Application Publication
(10) Pub. No.: US 2026/0215662 A1
(43) Pub. Date: Jul. 30, 2026

(54) ARTICULATION JOINTS AND ASSOCIATED ASSEMBLIES FOR MEDICAL DEVICES

(71) Applicant: Boston Scientific Scimed Inc., Maple Grove, MN (US)

(72) Inventors: Neal ANDERSON, Northborough, MA (US); Sean POWELL, Holden, MA (US); Brent DOLAN, Blaine, MN (US); Jordan HANAHAN, Natick, MA (US); Jared ROBERTSON, Somerville, MA (US); Brian Paul EDISON, Otsego, MN (US)

(73) Assignee: Boston Scientific Scimed Inc., Maple Grove, MN (US)

(21) Appl. No.: 19/460,598

(22) Filed: Jan. 27, 2026

Related U.S. Application Data
(60) Provisional application No. 63/750,995, filed on Jan. 29, 2025.

(51) Int. Cl. A61B 1/00; A61B 1/005

(52) U.S. Cl. CPC A61B 1/00128; A61B 1/00133; A61B 1/0052

(57) ABSTRACT

A medical device may comprise a handle, a shaft extending from the handle, an adapter fixed to a distal end of the shaft, and an articulation joint. The adapter may have a proximal portion and a distal portion. The distal portion may include a rib extending approximately parallel to a longitudinal axis of the shaft and a locking tab. The articulation joint may have a proximal-most link. The proximal-most link may include a proximal cavity, an opening on a radially outer surface of the proximal-most link, and a cutout. The proximal cavity may be configured to receive the distal portion of the adapter. The opening may be configured to receive the locking tab of the adapter. The cutout may be configured to receive the rib of the adapter.

### CROSS-REFERENCE TO RELATED APPLICATIONS

This application claims the benefit of priority from U.S. Provisional Application No. 63/750,995, filed on January 29, 2025, which is incorporated by reference herein in its entirety.

### FIELD OF INVENTION

The disclosure relates generally to endoscopic medical devices and assemblies. More particularly, in some embodiments, the disclosure relates to articulation joints and associated assemblies for endoscopes and other medical instruments. In some aspects, the disclosure describes adapters that may connect articulation joints to shafts of medical devices.

### BACKGROUND

Endoscopes and similar medical devices generally include a handle and an insertion portion. The insertion portion includes a flexible shaft, a working distal tip, and an articulation joint joining the working tip and the flexible shaft. The flexible shaft and the articulation joint are frequently covered by an outer sheath. The articulation joint often includes a plurality of links. Steering wires coupled to the articulation joint and to actuators of the handle may facilitate steering of the insertion portion by using (e.g., rotating) the actuators of the handle.

### SUMMARY

According to an example, a medical device may comprise a handle, a shaft extending from the handle, an adapter fixed to a distal end of the shaft, and an articulation joint. The adapter may have a proximal portion and a distal portion. The distal portion may include a rib extending approximately parallel to a longitudinal axis of the shaft and a locking tab. The articulation joint may have a proximal-most link. The proximal-most link may include a proximal cavity, an opening on a radially outer surface of the proximal-most link, and a cutout. The proximal cavity may be configured to receive the distal portion of the adapter. The opening may be configured to receive the locking tab of the adapter. The cutout may be configured to receive the rib of the adapter.

Any of the devices disclosed herein may include any of the following features, alone or in any combination. The medical device may further include a steering wire that extends through the shaft. The adapter may further comprise a groove disposed on an internal surface of the distal portion. The steering wire may be received in the groove. The groove may be circumferentially aligned with the rib. The groove may taper to a point at a proximal end. A radial depth of the groove may increase from the proximal end to a distal end of the groove.

The rib may be one or four ribs distributed circumferentially around the distal portion. The locking tab may be disposed on a radially outer surface of the rib. The locking tab may be configured to deflect during insertion of the distal portion of the adapter into the proximal cavity of the proximal-most link and return to an un-deflected position when aligned with the opening.

The adapter may be overmolded directly onto the distal end of the shaft. The adapter may include a ledge between the proximal portion and the distal portion. The distal end of the shaft may terminate proximally of the ledge. A proximal-most edge of the proximal-most link may abut the ledge.

The proximal-most link may include an internal wall. The internal wall may be distal of a proximal-most end of the proximal-most link. The internal wall may define a distal-most end of the proximal cavity.

The cutout may be circumferentially aligned with a channel extending through the articulation joint. The opening may be circumferentially aligned with the cutout.

The locking tab may be a first locking tab. The articulation joint may further comprise a distal-most link. The distal-most link may include a second locking tab extending radially inward relative to an internal surface of the distal-most link.

According to another example, a medical device may include a handle, a shaft extending distally from the handle, an articulation joint comprising a plurality of links, and an adapter connected a distal end of the shaft to the articulation join. The articulation joint may include a proximal-most link having a proximal cavity. The adapter may include a proximal portion coupled to the distal end of the shaft and a distal portion received within the proximal cavity of the proximal-most link. The distal portion may include a protrusion extending radially outward. The proximal-most link of the articulation joint may include an opening configured to receive the protrusion of the adapter. The opening and the protrusion may cooperate to resist rotation between the adapter and the articulation joint.

Any of the devices disclosed herein may include any of the following features, alone or in any combination. The distal portion of the adapter includes a rib extending radially outward from an outer surface of the distal portion. The rib may engage with an inner surface of the proximal cavity of the proximal-most link.

The medical device may further comprise a steering wire. The adapter may further comprise a groove disposed on an internal surface of the distal portion. The groove may receive the steering wire. The groove may taper to a point at a proximal end of the groove. A radial depth of the groove may increase from the proximal end to a distal end of the groove.

According to another example, a medical adapter may comprise a proximal portion configured to couple to a distal end of the shaft and a distal portion configured to be received within a proximal cavity of an articulation joint. The distal portion may include a locking tab disposed on a radially outer surface of the distal portion. A groove may be disposed on an internal surface of the distal portion. The groove may be configured to accommodate a steering wire. The groove may be circumferentially aligned with a rib. The groove may taper to a point at a proximal end of the groove. A radial depth of the groove may increase from the proximal end to the distal end of the groove.

The foregoing general description of the illustrative embodiments and the following detailed description thereof are merely exemplary aspects of the teachings of this disclosure and are not restrictive.

### BRIEF DESCRIPTION OF FIGURES

The accompanying drawings, which are incorporated and constitute a part of this specification, illustrate various exemplary embodiments and, together with the description, serve to explain the principles of the disclosed embodiments.

FIG. 1 illustrates an exemplary medical device, according to aspects of the disclosure.

FIG. 2 illustrates a perspective view of a distal end portion of the medical device of FIG. 1, according to aspects of the disclosure.

FIG. 3. illustrates a side view of an articulation joint of the distal end portion of FIG. 2, according to aspects of the disclosure.

FIG. 4 illustrates another perspective view of the articulation joint, according to aspects of the disclosure.

FIG. 5 illustrates a front view of a distal-most link of the articulation joint, according to aspects of the disclosure.

FIG. 6 illustrates view of a distal portion of the of the articulation joint, showing elements extending through the articulation joint in cross-section, according to aspects of the disclosure.

FIG. 7 illustrates another perspective view of the articulation joint, according to aspects of the disclosure.

FIG. 8 illustrates a back view of a proximal-most link of the articulation joint, according to aspects of the disclosure.

FIGS. 9 and 10 each illustrate a perspective view of a distal end of a shaft and an adapter, according to aspects of the disclosure.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. In some drawings, arrows labeled “P” and “D” indicate proximal and distal directions, respectively. The term “exemplary” is used in the sense of “example,” rather than “ideal.” The term “approximately,” or like terms (e.g., “substantially”), includes values +/- 10% of a stated value.

Articulation joints assist medical professionals to navigate complex anatomical structures and access hard-to-reach areas during diagnostic and therapeutic procedures. Improvements to articulation joints may contribute to enhanced device performance and increased procedural and manufacturing efficiency. The disclosed devices and assemblies provide an articulation joint comprised of a single component. For example, the articulation joint may include multiple portions integrally formed with one another. The articulation joint may include features to assist with securely coupling the articulation joint to the distal tip and/or to a distal end of the shaft. In these aspects, an adapter may assist in creating a secure connection between the articulation joint and a distal end of a flexible shaft (e.g., a flexible tube).

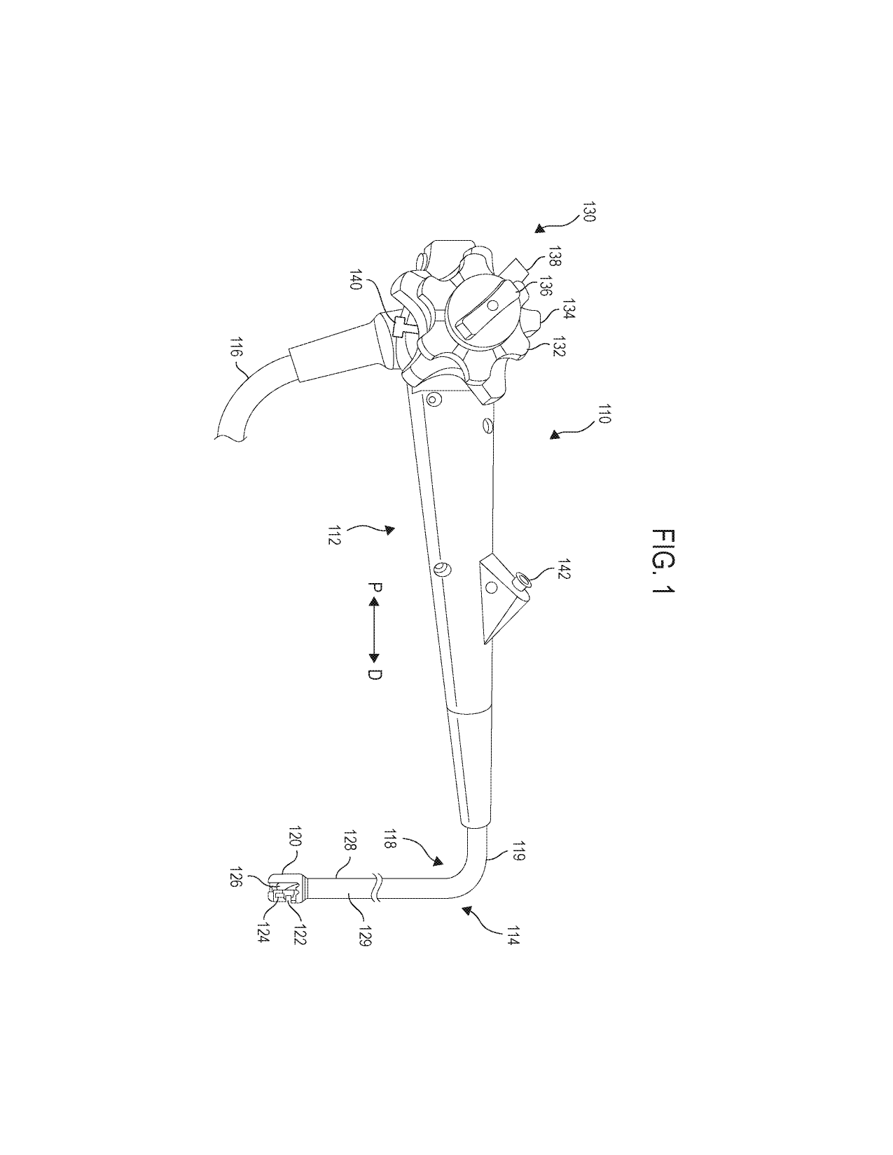

FIG. 1 depicts an exemplary medical device 110 having a handle 112 and an insertion portion 114. Medical device 110 may also include an umbilicus 116 for purposes of connecting medical device 110 to sources of, for example, air, water, suction, power, etc., as well as to image processing and/or viewing equipment. Although duodenoscopes and endoscopes are particularly referenced herein, the disclosure also encompasses other types of devices, such as bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices having an insertion portion configured for insertion into a subject’s body. A reference to an endoscope herein should be understood to encompass any of the above medical devices.

Insertion portion 114 may include a sheath or shaft 118 and a distal tip 120. Distal tip 120 may include an imaging device 122 (e.g., a camera) and a lighting element 124 (e.g., a light emitting diode (LED) or an optical fiber). Although the term “lighting element” is used herein, it will be appreciated that the term “lighting element” may include a plurality of lighting elements (e.g., a plurality of LEDs or optical fibers). Distal tip 120 may be side facing. That is, imaging device 122 and lighting element 124 may face radially outward, perpendicularly, approximately perpendicularly, or otherwise transverse to a longitudinal axis of shaft 118 and distal tip 120 (e.g., at an angle of approximately 70 degrees to an angle of approximately 100 degrees relative to a longitudinal axis of shaft 118). However, the disclosure is not limited to such an arrangement.

Distal tip 120 may also include an elevator 126 for changing an orientation of an accessory device or a tool inserted in a working channel of medical device 110. Elevator 126 may alternatively be referred to as a swing stand, pivot stand, raising base, or any suitable other term. Elevator 126 may be pivotable via, e.g., an actuation wire or another control element that extends from handle 112, through shaft 118, to elevator 126.

A distal portion of shaft 118 that is connected to distal tip 120 may include a steerable section 128. Shaft 118 may include a variety of structures that are known or may become known in the art. Described in further detail below, steerable section 128 may comprise an articulation joint assembly having at least four degrees of freedom (that is steerable in at least four directions). A flexible sheath or cover 129 may encompass, or surround, the articulation joint assembly of steerable section 128. In the following figures, the articulation joint assembly is shown with cover 129 removed.

Handle 112 may have one or more actuators/control mechanisms 130. One or more control mechanisms 130 may provide control over steerable section 128. One or more other control mechanisms 130 may allow for provision of air, water, suction, etc. For example, handle 112 may include control knobs 132, 134 for left, right, up, and/or down control of steerable section 128. For example, one of knobs 132, 134 may provide left/right control of steerable section 128, and the other of knobs 132, 134 may provide up/down control of steerable section 128. Although not shown in FIG. 1, handle 112 may include additional actuators/control mechanisms 130. The additional actuators/control mechanisms 130 may be configured to control additional aspects of medical device 110 (e.g., turning on/off imaging device 122 and/or lighting element 124, capturing an image or video via imaging device 122, etc.).

A plurality of actuation elements, such as cables or wires suitable for medical procedures (e.g., medical grade plastic or metal), may extend from handle 112, through shaft 118 to steerable section 128. In some examples, at least some of the cables or wires may extend to distal tip 120. Some of the cables or wires may be fixedly coupled to the articulation joint assembly.

Handle 112 may further include one or more locking mechanisms 136, 138 (e.g., knobs or levers) for preventing steering and/or braking of steerable section 128 in at least one of an up, down, left, or right direction. Handle 112 may include an elevator control lever 140. Elevator control lever 140 may raise and/or lower elevator 126, via a connection between lever 140 and an elevator control element. The elevator control element configured to raise and/or lower elevator 126 may extend from lever 140, through shaft 118 and steerable section 128, and to elevator 126. A port 142 may allow passage of a tool into a working channel of the medical device 110, to distal tip 120.

In use, an operator may insert at least a portion of shaft 118 into a body lumen of a subject. Distal tip 120 may be navigated to a procedure site in the body lumen. For example, the operator may push or urge distal tip 120 distally in order to advance distal tip 120 through the body lumen. The operator may insert an accessory device (not shown) into port 142, and pass the accessory device through shaft 118 via the working channel to distal tip 120. The accessory device may exit the working channel at distal tip 120. The user may use elevator control lever 140 to raise elevator 126 and angle the accessory device toward a desired location (e.g., a papilla of the pancreatico-biliary tract). The user may use the accessory device to perform a medical procedure. The user may rotate one or more actuators (e.g., control knobs 132, 134, etc.), to bend, or articulate, steerable section 128 in one or more directions.

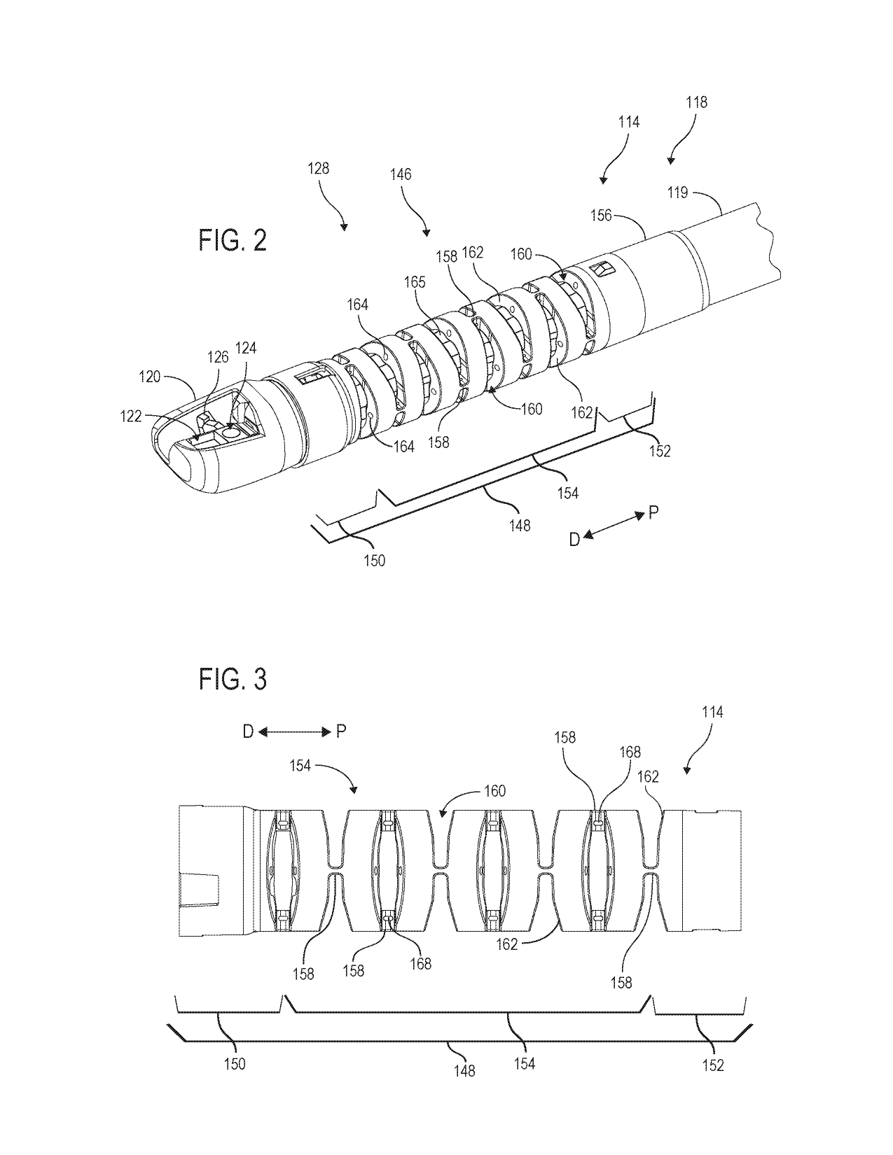

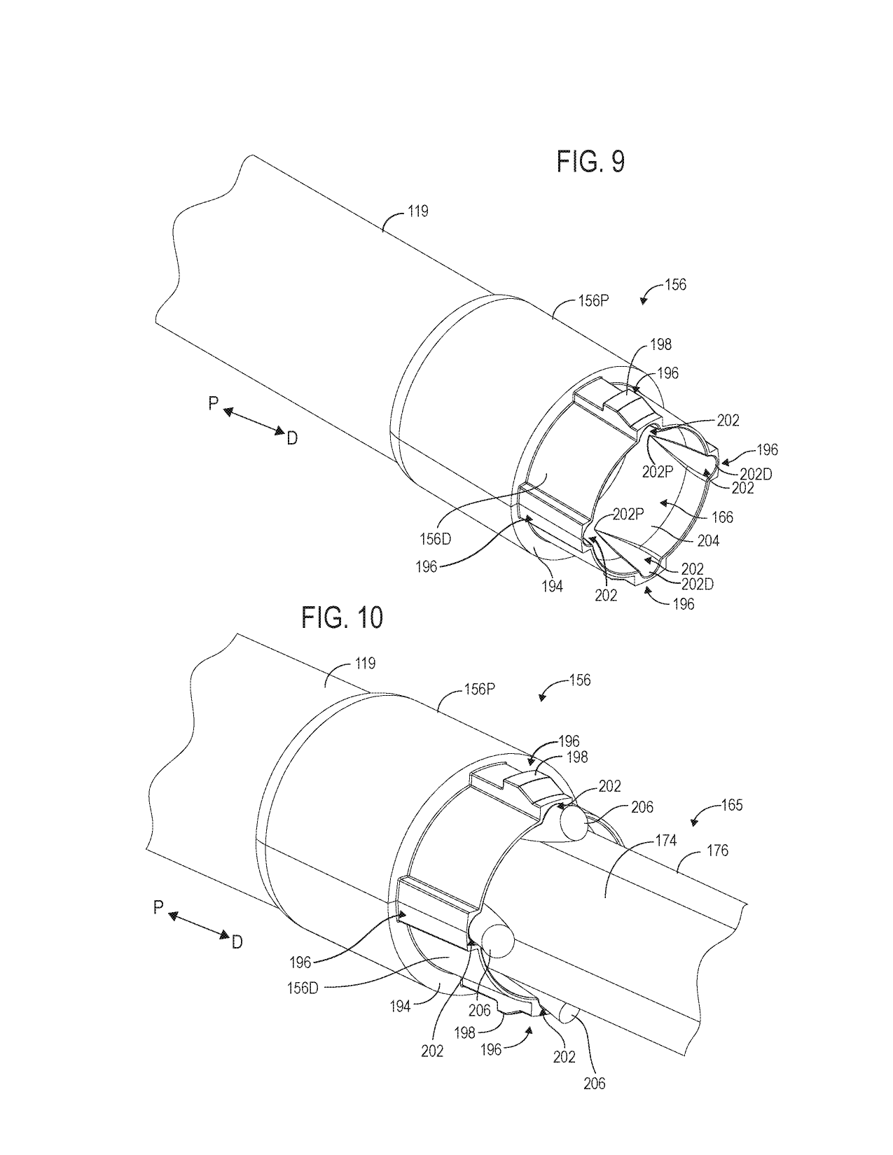

FIG. 2 illustrates a distal end portion of insertion portion 114 of medical device 110. For example, FIG. 2 illustrates a distal end of shaft 118, including a distal end of tubular member 119, distal tip 120, and steerable section 128 with cover 129 removed. As previously described, distal tip 120 may include an imaging device 122, a lighting element 124, and an elevator 126. Steerable section 128 may include an articulation joint 146. Articulation joint 146 may include a plurality of links 148. Plurality of links 148 may include a distal-most link 150, a proximal-most link 152, and a plurality of central links 154 disposed therebetween. Articulation joint 146 may have any suitable number of central links 154. Distal-most link 150 may be configured to couple to a distal tip 120. Proximal-most link 152 may be configured to couple to a distal-most end of a tubular member 119 (which may also be referred to as a shaft). In particular, proximal-most link 152 may be coupled to tubular member 119 via an adapter 156. In aspects, tubular member 119 may be a flexible tube that has a wall with a solid cross-section. Tubular member 119 may be passively bendable but not actively steerable. Aspects of adapter 156 are described in further detail below with respect to FIGS. 9 and 10.

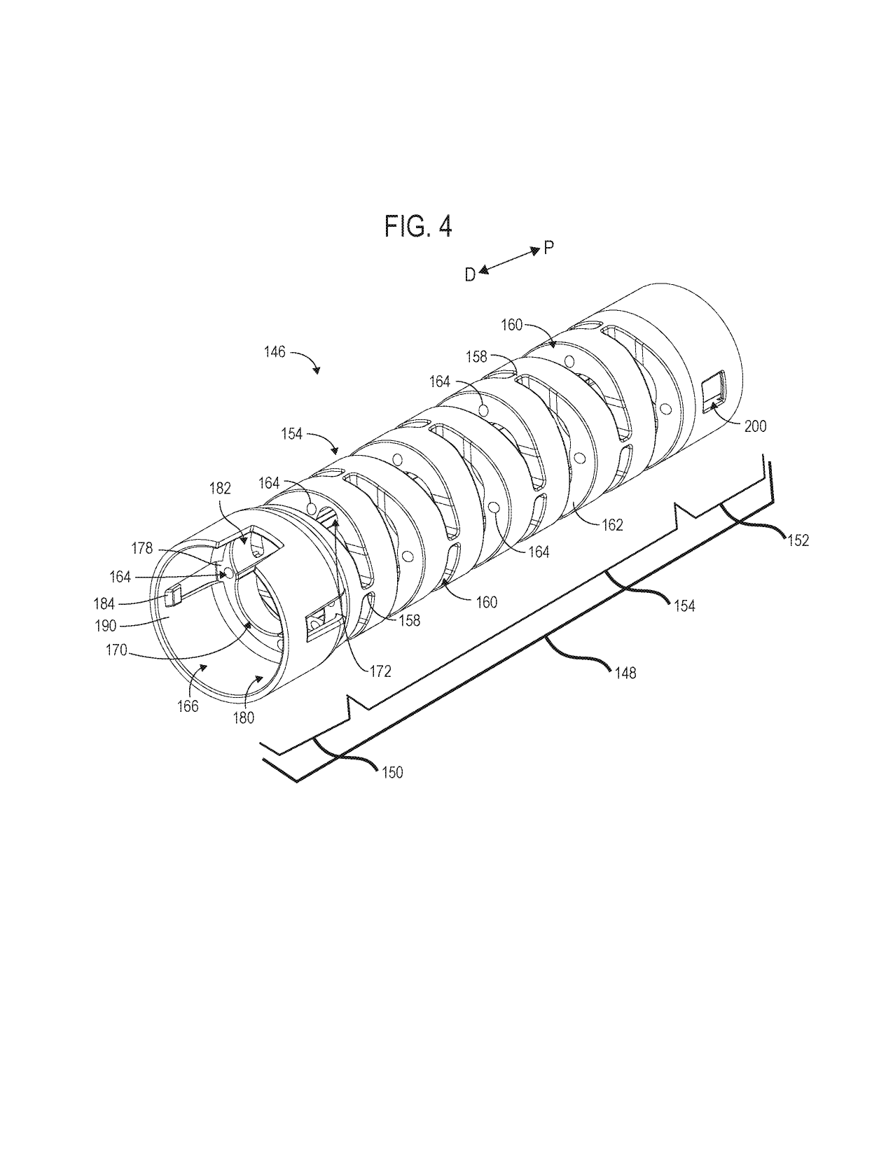

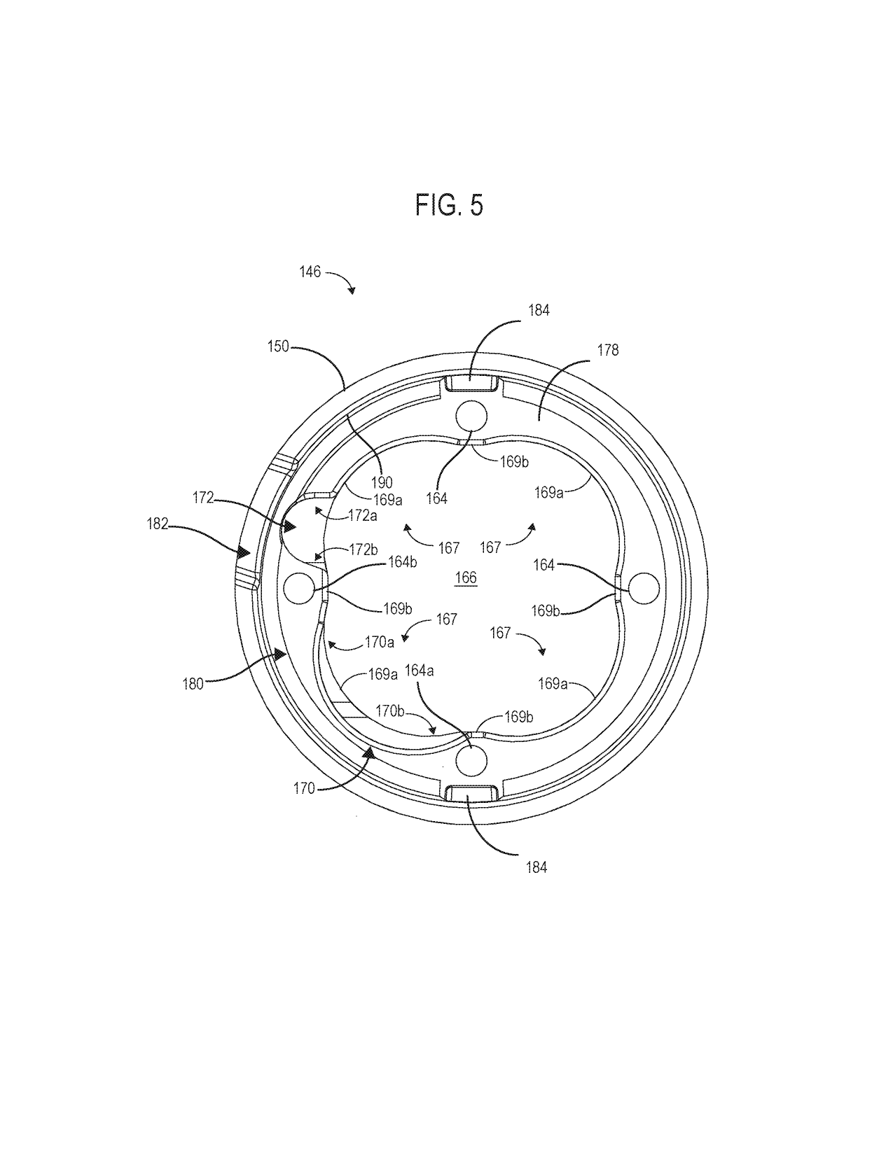

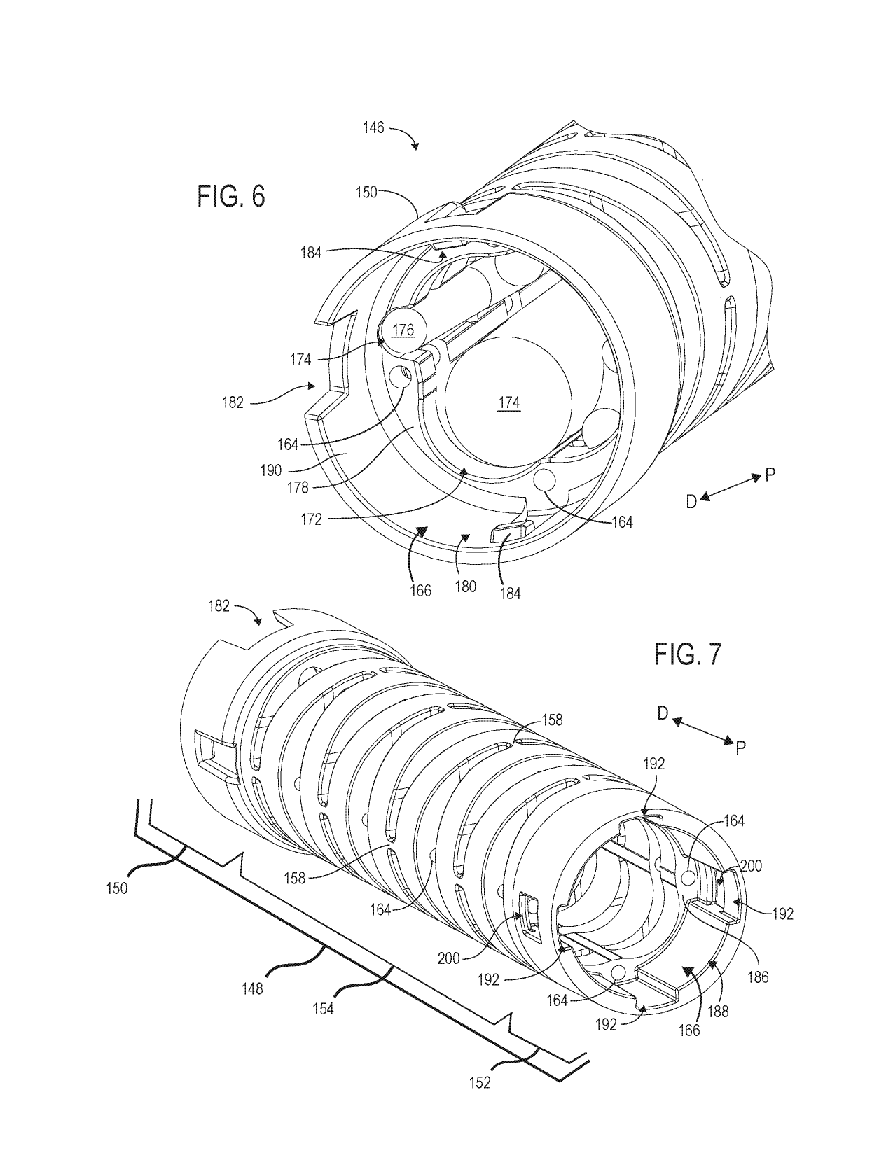

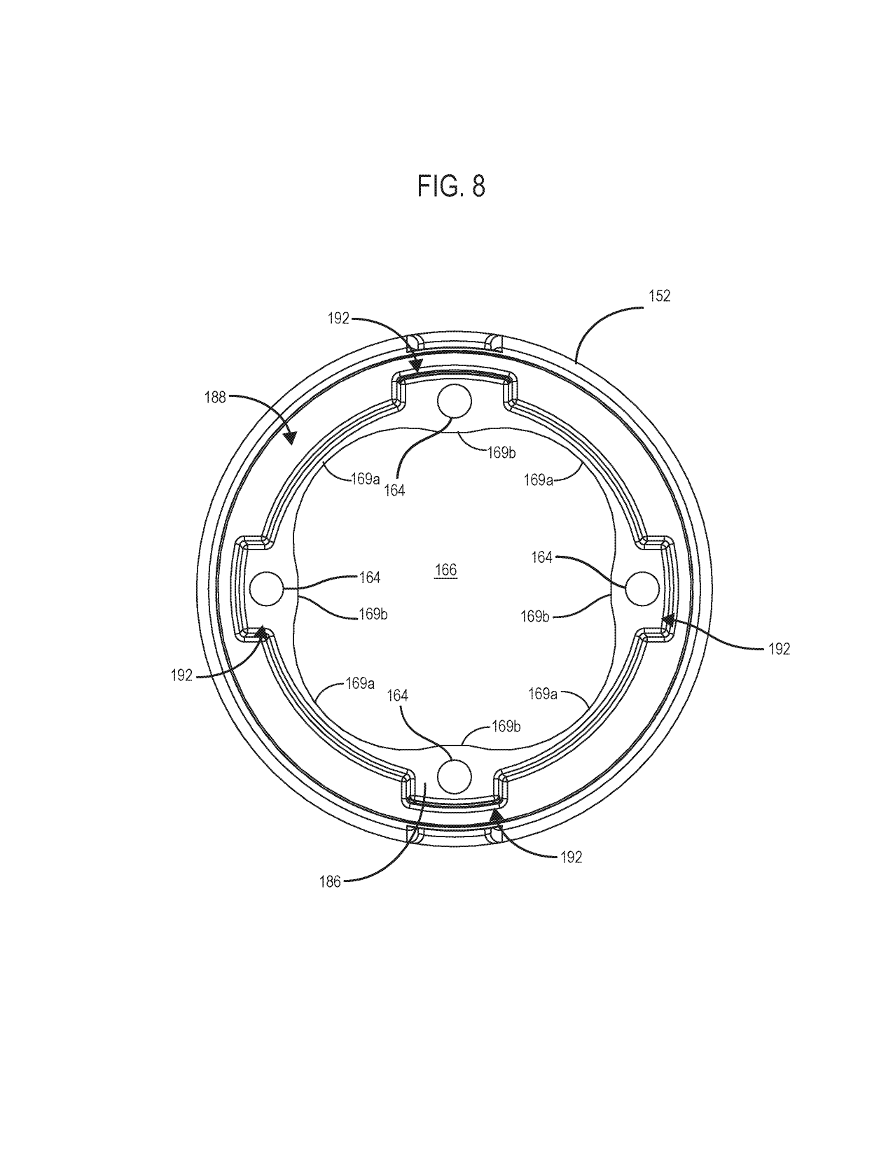

Aspects of articulation joint 146 are shown and described in further detail below with respect to FIGS. 3-8. In particular, FIG. 3 illustrates a side view of articulation joint 146; FIG. 4 illustrates a perspective view of articulation joint 146 with distal-most link 150 at the bottom left corner of the figure and proximal-most link 152 at the top right corner of the figure; FIG. 5 illustrates a distal-to-proximal facing view of distal-most link 150 of articulation joint 146; FIG. 6 illustrates a perspective view of a distal portion of articulation joint 146 with elements of medical device 110 that extend through articulation joint 146 shown in cross-section; FIG. 7 illustrates an alternative perspective view of articulation joint 146 with distal-most link 150 at the top left corner of the figure and proximal-most link 152 at the bottom right corner of the figure; and FIG. 8 illustrates a proximal-to-distal facing view of proximal-most link 152. FIGS. 2-8 may be referred to interchangeably throughout the following description.

Referring to FIGS. 2-8, distal-most link 150, proximal-most link 152, and each link of plurality of central links 154 may be connected to adjacent link(s) by at least two living hinges 158 (also referred to, or known as, integral hinges). In some aspects, adjacent links (e.g., distal-most link 150, proximal-most link 152, and each link of the plurality of central links 154) may be integrally formed via living hinges 158. For example, an entirety of articulation joint 146 may be a single molded, or formed, component. In aspects, living hinges 158 may provide flexibility to articulation joint 146. For example, living hinges 158 may enable articulation joint 146 to bend and articulate in multiple directions (e.g., left, right, up, and/or down). Living hinges 158 may be formed as thin portions of material extending between the more rigid adjacent structures of links 150, 152, 154.

Adjacent links may be connected by a pair of living hinges 158. The living hinges 158 of the pair of hinges 158 may be spaced approximately 180 degrees from one another. In some cases, a radially outer surface of each living hinge 158 may be aligned with a radially outer surface of each adjacent link. In these aspects, the radially outer surfaces of each adjacent link and each living hinge 158 extending therebetween may be continuous. This arrangement of living hinges 158 may allow for controlled articulation of articulation joint 146 in multiple directions. For example, the paired living hinges 158 positioned opposite each other may facilitate bending of articulation joint 146 in a plane that is transverse to a plane extending through the living hinges 158 of a given pair.

A position of adjacent pairs of living hinges 158 may alternate to provide steerability in three or four directions. For example, hinges 158 of a first pair of living hinges 158 may be arranged at positions corresponding to 12 o'clock and 6 o'clock on the circumference of articulation joint 146. Hinges of an adjacent, second pair of living hinges 158 may be arranged at 3 o'clock and 9 o'clock. In other words, adjacent pairs of living hinges 158 may be offset from one another by 90 degrees. This alternating pattern of the hinge placement may continue along a length of articulation joint 146, allowing for articulation joint 146 to have four directions of motion (e.g., left, right, up, down directions). This arrangement may allow articulation joint 146 to perform complex maneuvers within constrained environments, such as within the human body during medical procedures.

Articulation joint 146 may include gaps 160 defined between adjacent links 148. Gaps 160 may allow articulation joint 146 to bend and flex during use. Gaps 160 may be defined by surfaces 162 of adjacent links 148 in articulation joint 146, and living hinges 158 may span gaps 160. In some cases, surfaces 162 may be angled to facilitate greater bending of articulation joint 146. For example, surfaces 162 defining gaps 160 may have a tapered or beveled shape, allowing adjacent links 148 to pivot relative to each other to a greater degree, inhibiting or delaying contact between adjacent surfaces 162. In other cases, surfaces 162 may define little to no angle, disallowing or limiting articulation in one direction and allowing bending of articulation joint in an opposite direction. This angled configuration of surfaces 162 may enable articulation joint 146 to achieve a tighter bending radius and/or more extreme articulation angles. In particular, a size of gaps 160 and an angle of surfaces 162 may be designed to allow for a desired range of motion while still maintaining structural integrity of articulation joint 146. In some implementations, the dimensions and geometry of gaps 160 may vary along the length of articulation joint 146 to provide different articulation characteristics in different sections of the joint.

When articulation joint 146 is in a fully bent configuration, surfaces 162 defining gaps 160 may optionally come into contact with each other. As articulation joint 146 bends, gaps 160 on one side may narrow while gaps 160 on the opposite side widen. In some cases, surfaces 162 of adjacent links on the narrowing side may abut or touch, providing a physical stop to prevent over-articulation of articulation joint 146. This contact between surfaces 162 of adjacent links may also help distribute forces along articulation joint 146 when in a bent configuration, potentially improving the overall strength and stability of the articulated position.

Articulation joint 146 may include a plurality of channels 164 configured to accommodate both passive and active actuation elements (e.g., wires, cables, or other elongated elements). For example, channels 164 may extend at least partially through each of links 148. These actuation elements may be cables, wires, or other suitable components capable of transmitting force along their length. By manipulating the actuation elements, such as shortening or lengthening them, articulation joint 146 may be articulated, or bent, in one or more directions. This configuration may allow for up/down and left/right articulation of articulation joint 146.

At least one actuation element may extend through each channel 164. Having actuation elements extend through channel 164 (as opposed to having the actuation elements extend through a central lumen 166 of articulation joint 146) may allow for additional space within central lumen 166 by reducing a number of components extending therethrough. In some cases, each link 148 of articulation joint 146, including distal-most link 150, proximal-most link 152, and each link of the plurality of central links 154, may include two or more (e.g., four) channels 164 extending proximally to distally therethrough. Each channel 164 extending longitudinally through each link 148 of articulation joint 146 may be parallel to a central longitudinal axis of articulation joint 146. In some implementations, channels 164 may be arranged symmetrically around central lumen 166 of articulation joint 146.

Central lumen 166 may be defined along a longitudinal length of articulation joint 146. A plurality of elements 165 may extend through central lumen 166. Plurality of elements 165 may include working channel 174, elevator actuation element 176, additional wires and/or cables, tubular members, etc. FIG. 3 shows four channels 164 positioned at approximately 90-degree intervals around central lumen 166 and extending through a proximal wall of distal-most link 150, and FIG. 4 shows four channels 164 positioned at approximately 90-degree intervals around central lumen 166 and extending through proximal-most link 152. Each link of central links 154 may have a similar arrangement of channels 164.

The number and positioning of channels 164 may vary depending on the desired degrees of freedom for articulation joint 146. In some cases, additional channels 164 may be included to provide enhanced articulation capabilities or to accommodate other elements such as optical fibers, fluid channels, or electrical wires. Channels 164 may be formed as integral features of each link during the manufacturing process (e.g., molding) of articulation joint 146. In some aspects, the inner surfaces of channels 164 may be smooth or coated to reduce friction and facilitate smooth movement of the actuation elements within the channels.

In some cases, channels 164 may be circumferentially aligned with living hinges 158 of articulation joint 146. For example, channels 164 may extend through living hinges 158. This alignment may allow for a continuous path for actuation elements through the articulation joint 146, including through living hinges 158. Actuation elements may also span gaps 160, for example, where hinges 158 are offset from the location of the actuation elements. Hinges 158 may be offset due to the alternating position of hinges 158 along a longitudinal length of articulation joint 146. Routing channels 164 through living hinges 158 may contribute to a more compact design of articulation joint 146, as channels 164 may utilize the existing flexible regions of the joint structure.

In some cases, living hinges 158 may include windows 168. Windows 168 may be formed in a surface that extends from a radially outer surface of articulation joint 146 to a radially inner surface of a given living hinge 158. The inclusion of windows 168 may provide additional flexibility to living hinges 158. Windows 168 may vary in size, shape, and arrangement along living hinges 158. For example, windows 168 may be circular, oval, rectangular, or any other suitable shape. In some implementations, the size and shape of windows 168 may be optimized to balance the flexibility and structural integrity of living hinges 158.

The design of channels 164 may also contribute to the overall flexibility and strength of articulation joint 146. The channels may be sized and shaped to maintain structural integrity of the links while allowing for the necessary range of motion. In some cases, the dimensions of channels 164 may vary along the length of articulation joint 146 to optimize performance characteristics in different sections of the joint.

Through proximal-most link 152 and central links 154, central lumen 166 may have a clover shape, as shown most clearly in FIG. 8. Walls of links 148 may include a plurality of lobes 167 (e.g., four lobes 167) that protrude radially inwardly toward a central longitudinal axis of articulation joint 146, thereby defining the clover shape of central lumen 166. Channels 164 may extend through lobes 167. The clover shape of central lumen 166 may maximize a size of central lumen 166. In aspects, the clover shape of central lumen 166 may assist in defining uniform walls for manufacturing articulation joint 146. Central lumen 166 may be defined by four concave surfaces 169a that curve away from the central longitudinal axis of central lumen 166 and by four convex surfaces 169b that curve in toward the central longitudinal axis of central lumen 166. Convex surfaces 169bmay extend along lobes 167. In some examples, a central longitudinal axis of central lumen 166 as it extends through proximal-most link 152 and central links 154 may be coaxial or approximately coaxial with the central longitudinal axis of articulation joint 146.

Shown more clearly in FIGS. 4-7, distal-most link 150 may include a first cutout 170 and a second cutout 172. First cutout 170 and second cutout 172 may be continuous with central lumen 166 within at least a portion of distal-most link 150. In some implementations, first cutout 170 and second cutout 172 may extend into the wall of distal-most link 150 that defines central lumen 166. In some aspects, the first cutout 170 and second cutout 172 may be only in distal-most link 150. In alternatives, first cutout 170 and second cutout 172 may extend into some or all of central links 154 and proximal-most link 152 (e.g., into only an adjacent link to distal-most link 150).

For example, first cutout 170 may be defined by a portion 171 of a wall 178 of distal-most link 150. As shown in FIG. 5, a first end 170a of first cutout 170 may be circumferentially overlapping with a first channel 164a of channels 164 (a bottom channel 164 as shown in FIG. 5). A second end 170bof first cutout 170 may not overlap with a second, adjacent channel 164b (a left channel 164 as shown in FIG. 5). Thus, first cutout 170 may be off-centered between adjacent channels 164 (offset toward first channel 164a).

Second cutout 172 may be defined by a portion 173 of wall 178. A first end 172a of second cutout 172 may be approximately circumferentially aligned with an outer surface of second channel 164b. In other words, a line drawn tangent to an edge of second channel 164b and extending perpendicularly to a central longitudinal axis of distal-most link 150 (i.e., a line extending across the top edge of second channel 164b in FIG. 5) may be at or very near to first end 172a. A second end 172b of second cutout 172 may be relatively close to first end 172a circumferentially. Second cutout 172 may be approximately symmetrical about a line extending through a center of second cutout 172. However, the line of symmetry of second cutout 172 may not be aligned with any radius of distal-most link 150 (a line extending between the central longitudinal axis of distal-most link 150 and a radially outer surface of distal-most link 150). Thus, second cutout 172 may be asymmetrical with respect to all radii of distal-most link 150.

A displacement between first end 170a and second end 170b of first cutout 170 may be larger than a displacement between first end 172a and second end 172b of second cutout 172. Second cutout 172 may be radially deeper than first cutout 170. In other words, a radially outermost edge of second cutout 172 may be closer to an outer surface of distal-most link 150 than a radially outermost edge of first cutout 170 is.

First cutout 170 and second cutout 172 may be configured to accommodate elements extending through articulation joint 146. For example, first cutout 170 may be sized and shaped to receive a working channel 174. Working channel 174 may extend from handle 112, through shaft 118 and articulation joint 146 and into distal tip 120. In some cases, second cutout 172 may be sized and/or shaped to receive an elevator actuation element 176. Elevator actuation element 176 may extend from handle 112, through shaft 118 and articulation joint 146, to elevator 126 in distal tip 120. Due to first cutout 170 and second cutout 172, a central channel (including central lumen 166, first cutout 170, and second cutout 172) of distal-most link 150 may be asymmetrical and larger than central lumen 166 as it extends through proximal-most link 152 and central links 154.

The inclusion of first cutout 170 and second cutout 172 in distal-most link 150 may help to align components of medical device 110 with related portions of distal tip 120. For example, first cutout 170 may receive a working channel 174. First cutout 170 may help to align working channel 174 with a portion of distal tip 120 to which working channel 174 is coupled. First cutout 170 may also encourage working channel 174 to adopt a preferred position in more proximal portions of shaft 118, including in proximal-most link 152 and/or central links 154. Second cutout 172 may accommodate an elevator actuation element 176 (e.g., wire or cable). Second cutout 172 may help to align elevator actuation element 176 with a coupling point on elevator 126. Second cutout 172 may also encourage elevator actuation element 176 to adopt a preferred position in more proximal portions of shaft 118, including in proximal-most link 152 and/or central links 154. A size and shape of cutouts 170, 172 may be chosen to accommodate outer surfaces of the relevant structures received therein (e.g., working channel 174 or elevator actuation element 176). The size and shape of cutouts 170, 172 may also be chosen so as to discourage other elements from entering cutouts 170, 172. Additionally or alternatively, cutouts 170, 172 may also provide additional space for working channel 174 and elevator actuation element 176 within distal-most link 150.

In some aspects, a distal-most central link of the plurality of central links 154 (e.g., a central link that is immediately proximal of distal-most link 150) or others of central links 154 may also include cutouts similar to first cutout 170 and second cutout 172. The inclusion of additional cutouts in the distal-most central link of the plurality of central links 154 may assist in creating a smooth transition of working channel 174 and elevator actuation element 176 into distal tip 120. For example, the inclusion of additional cutouts in the distal-most central link of the plurality of central links 154 may prevent working channel 174 and elevator actuation element 176 from having a sharp bend as working channel 174 and elevator actuation element 176 extend into distal tip 120. In some aspects, cutouts in central link(s) 154 may have tapering depths to transition to the final depth of cutouts 170 and 172.

In some aspects, cutouts 170, 172 may extend along an entire length of articulation joint 146, thereby increasing a space of central lumen 166 that is available to other elements. In some aspects, cutout 170 may allow use of a larger working channel 174.

Distal-most link 150 may include recessed wall 178 that is proximal of a distal end of distal-most link 150. Recessed wall 178 may define a proximal end of a distal cavity 180. Distal cavity 180 may be configured to receive a proximal portion of distal tip 120. This configuration may allow for a smooth transition between articulation joint 146 and distal tip 120 (e.g., a continuous outer surface between articulation joint 146 and distal tip 120).

A distal opening of each channel 164 extending through distal-most link 150 may be disposed on a distal surface of recessed wall 178. A distal-most end of the actuation elements extending through articulation joint 146 may terminate distally of the distal opening of each channel 164. For example, the distal-most end of the actuation elements extending through each channel 164 may terminate within distal cavity 180, for example, proximal of distal tip 120. Alternatively, the actuation elements may extend into distal tip 120. In some cases, a depth of distal cavity 180 (e.g., measured from the distal-most end of distal-most link 150 to recessed wall 178) may be varied to accommodate different configurations or sizes of distal tip 120 and/or different configurations or sizes of the distal-most ends of each actuation element.

Distal-most link 150 may include a keying feature 182. Keying feature 182 may be configured to receive a corresponding key of distal tip 120. In some cases, keying feature 182 may be formed as a cut out (as shown), a protrusion, or a recess on an inner surface of distal-most link 150. The shape and size of keying feature 182 may be designed to mate with a complementary feature on distal tip 120 (e.g., a complementary recess or protrusion). Keying feature 182 may facilitate proper alignment between distal-most link 150 and distal tip 120 during assembly or connection of these components. In aspects, keying feature 182 may ensure that distal tip 120 is oriented in a specific rotational position relative to distal-most link 150.

In some aspects, keying feature 182 may have an asymmetric shape that corresponds to a unique shape of distal tip 120. This configuration may prevent incorrect assembly by allowing distal tip 120 to be attached to distal-most link 150 in only one specific orientation. Keying feature 182 may also provide a physical stop that prevents over-insertion of distal tip 120 into distal-most link 150. Keying feature 182 may additionally serve to transfer torque between articulation joint 146 and distal tip 120. This torque transfer may be important for maintaining the rotational position of distal tip 120 during use of medical device 110, particularly when articulation joint 146 is bent or articulated. In some examples, keying feature 182 may assist with fixing distal tip 120 to distal-most link 150.

Distal-most link 150 may include at least one locking tab 184 (e.g., two locking tabs 184, as shown). Locking tab 184 may extend radially inward relative to an internal surface 190 of distal cavity 180. In some cases, locking tab 184 may be configured to interact with a receiving feature on distal tip 120, for example, to securely couple distal-most link 150 with distal tip 120. For example, the inclusion of locking tab 184 may provide a secure mechanical connection between articulation joint 146 and distal tip 120. In some implementations, locking tab 184 may be formed as an integral part of distal-most link 150. The radially inward extension of locking tab 184 may allow locking tab 184 to engage with a corresponding groove, recess, or other receiving feature on the proximal portion of distal tip 120 when the two components are brought together.

In some cases, locking tab 184 may be designed with a slight flexibility and may be resilient. This property may allow locking tab 184 to deflect slightly during the insertion of distal tip 120 into distal cavity 180, and then snap back into place (e.g., return to an un-deflected position) once aligned with the receiving feature on distal tip 120. This configuration may provide an audible or tactile indication to the user that proper coupling has been achieved. The shape and size of locking tab 184 may be varied to accommodate different coupling requirements or to provide different levels of retention force. For example, locking tab 184 may have a rounded or chamfered leading edge to facilitate smooth insertion of distal tip 120. In some implementations, multiple locking tabs 184 may be distributed around the circumference of distal cavity 180 to provide a more secure connection. For example, as shown more clearly in FIG. 3, distal-most link 150 may include at least two locking tabs 184 arranged opposite from one another.

In some aspects, locking tab 184 may work in conjunction with other coupling features, such as keying feature 182, to provide a robust and precisely aligned connection between articulation joint 146 and distal tip 120. This combination of coupling mechanisms may enhance the overall stability and functionality of medical device 110 during use. In some aspects, no glue may be used to couple distal tip 120 to distal-most link 150, and only the mechanical couplings discussed above may fix distal tip 120 to distal-most link 150.

Shown more clearly in FIGS. 7 and 8, proximal-most link 152 may include an internal wall 186. For example, internal wall 186 may be distal of a proximal-most end of proximal-most link 152. A proximal opening of each channel 164 may be disposed on a proximal face of wall 186. Internal wall 186 may define a distal end of a proximal cavity 188. Proximal cavity 188 may be configured to receive a distal end of adapter 156.

Proximal-most link 152 may further include wall cutouts 192, which may be formed in a radially outer wall of proximal link 152. In some cases, cutouts 192 may only be present in proximal-most link 152. Cutouts 192 may be circumferentially aligned with channels 164. Wall cutouts 192 may be configured to receive complementary ribs 196 on adapter 156 (FIGS. 9 and 10), described in further detail below. This configuration may allow for proper alignment and a secure connection between proximal-most link 152 and adapter 156.

In some aspects, proximal-most link 152 may include two or more wall grooves or cutouts 192. The number of wall cutouts 192 may correspond to the number of channels 164 in articulation joint 146. For example, if articulation joint 146 includes four channels 164, proximal-most link 152 may include four wall cutouts 192. Similarly, if articulation joint 146 includes two channels 164, proximal-most link 152 may include two wall cutouts 192. In other aspects, the number of wall cutouts 192 may be greater than the number of channels 164 to provide fewer or additional alignment options and/or to accommodate different configurations of adapter 156.

Wall cutouts 192 may have a generally rectangular or elongated cross-sectional shape. Wall cutouts 192 may extend along at least a portion of an axial length of proximal-most link 152. In some implementations, the width of wall cutouts 192 may be sized to accommodate complementary ribs 196 on adapter 156, allowing for a snug fit between the components. The shape and size of wall cutouts 192 may vary depending on the specific design requirements of articulation joint 146 and adapter 156. For example, in some cases, wall cutouts 192 may have a tapered or angled profile to facilitate easier insertion of ribs 196 during assembly. The depth of wall cutouts 192 may also be adjusted to provide the desired level of engagement between proximal-most link 152 and adapter 156.

Proximal-most link 152 may include an opening 200. Opening 200 may extend through radially outer walls of proximal-most link 152. In some cases, openings 200 may be circumferentially aligned with wall cutouts 192 of proximal-most link 152. For example, opening 200 may be configured to receive a protrusion, such as a locking feature of adapter 156 (e.g., a locking tab 198 of adapter 156, discussed below). In these aspects, opening 200 and locking tab 198 may cooperate to resist rotation of articulation joint 146 and adapter 156. The alignment of openings 200 with wall cutouts 192 may provide a more secure connection between proximal-most link 152 and adapter 156, as the features of adapter 156 may engage with both structural elements. This configuration may enhance the overall stability of the connection between articulation joint 146 and adapter 156, potentially improving the performance and reliability of medical device 110 during use. The arrangement of internal wall 186, proximal cavity 188, wall cutouts 192, and opening 200 in proximal-most link 152 may contribute to the overall functionality and assembly of articulation joint 146. These features may allow for secure attachment to adapter 156 while maintaining the flexibility and articulation capabilities of articulation joint 146.

Referring to FIGS. 9 and 10, adapter 156 may include a proximal portion 156P and a distal portion 156D. In some aspects, proximal portion 156P may include a larger diameter as compared to distal portion 156D. Distal portion 156D may be received within opening 200 of proximal-most link 152. At a transition point between proximal portion 156P and distal portion 156D, a ledge 194 may be defined. During assembly, a proximal-most end (e.g., proximal-most edge) of articulation joint 146 may abut ledge 194. Ledge 194 may prevent distal portion 156D from being over-inserted into proximal cavity 188 of proximal-most link 152. In aspects, shaft 118 (e.g., tubular member 119) may terminate proximally of ledge 194.

Distal portion 156D may include at least one rib 196 protruding radially outward relative to a surrounding radially outer surface of distal portion 156D. In some implementations, distal portion 156D may include four ribs 196, as shown in FIGS. 8 and 9. However, distal portion 156D may include fewer or additional ribs 196 in other cases. Ribs 196 may be received by wall cutouts 192 of proximal-most link 152 during assembly. Each rib 196 may extend approximately parallel to the longitudinal axis of shaft 118, including tubular member 119, and a locking tab 198 of adapter 156. Ribs 196 may be disposed circumferentially around distal portion 156D of adapter 156.

Each rib 196 may include an element groove 202 disposed on an internal surface 204 of distal portion 156D. In some aspects, each element groove 202 may be formed as rounded, oval-like groove that tapers to a point 202P at a proximal end and have a wider distal end 202D. As each element groove 202 widens moving in a distal direction, a depth (in a radial direction, perpendicular to a central longitudinal axis of adapter 156) of the groove within the wall defining distal portion 156D may increase. Element grooves 202 may be configured to accommodate and may receive steering elements 206 (e.g., steering wires and/or Bowden cables), which may exit tubular member 219, extend through adapter 156, and pass into channels 164 of articulation joint 146. The configuration of grooves 202 may facilitate directing steering elements 206 into channels 164. Grooves 202 may also help to maintain separation among steering elements 206 within tubular member 219.

In an alternative exemplary configuration, proximal-most link 152 may include rib(s), similar to rib(s) 196, described above. In such a configuration, adapter 156 (e.g., distal portion 156D of adapter 156) may include cutouts, similar to wall cutouts 192, described above with respect to FIG. 8. In these aspects, the cutouts of adapter 156 may be configured to receive the rib(s) of proximal-most link 152.

In a further alternative configuration, each of proximal-most link 152 and adapter 156 may include a combination of rib(s) and cutout(s). For example, proximal-most link 152 may include a cutout configured to receive a rib of adapter 156 in addition to a rib configured to be received by a cutout of adapter 156. The rib(s) and cutout(s) may be arranged in any order on proximal-most link 152 and/or adapter 156. For example, the rib(s) and cutout(s) may be arranged on proximal-most link 152 in an alternating pattern (e.g., a first rib, a first cutout, a second rib, etc.) or in any combination (e.g., a first rib, a second rib, a first cutout, etc.). Accordingly, the cutout(s) and rib(s) may be arranged on adapter 156 in a corresponding manner (e.g., such that each rib of proximal-most link 152 is received by a corresponding cutout of adapter 156, and such that each cutout of proximal-most link 152 is configured to receive a corresponding rib of adapter 156).

As described, adapter 156 may further include locking tab 198 (e.g., a proximal locking tab 198 for securing a proximal end of articulation joint 146) positioned on distal portion 156D. In particular, proximal locking tab(s) 198 may be disposed on a radially outer surface of at least one of ribs 196. In some implementations, proximal locking tabs 198 may be received by openings 200 of proximal-most link 152 of articulation joint 146. Proximal locking tabs 198 and ribs 196 may work together to provide a secure connection between adapter 156 and articulation joint 146. In some cases, ribs 196 may help retain articulation joint 146 in position, particularly when subjected to torque forces.

In some implementations, adapter 156 may be designed to connect tubular member 119 to articulation joint 146. Proximal portion 156P may be configured to fit over the distal end of tubular member 119. For example, tubular member 119 may be received within a lumen of proximal portion 156P. Distal portion 156D may be designed to connect with articulation joint 146 via the mechanisms described above. This configuration may allow for a secure connection between the shaft 118 and articulation joint 146, while also providing pathways for steering elements 206 and other internal components to pass through adapter 156 and into articulation joint 146.

Similar to distal locking tab 184, proximal locking tab 198 may be designed with a slight flexibility and may be resilient. This property may allow proximal locking tab 198 to deflect slightly during the insertion of adapter 156 into proximal-most link 152, and then snap back into place (e.g., return to an un-deflected position) once aligned with opening 200 on proximal-most link 152. This configuration may provide an audible or tactile indication to the user that proper coupling has been achieved between adapter 156 and articulation joint 146.

The shape and size of proximal locking tab 198 may be varied to accommodate different coupling requirements or to provide different levels of retention force. For example, proximal locking tab 198 may have a rounded or chamfered leading edge to facilitate smooth insertion into opening 200 of proximal-most link 152. In some implementations, multiple proximal locking tabs 198 may be distributed around the circumference of distal portion 156D to provide a more secure connection. For example, as shown in FIGS. 8 and 9, adapter 156 may include at least two proximal locking tabs 198 arranged diametrically opposite from one another.

In some aspects, element grooves 202 of adapter 156 may be circumferentially aligned with channels 164 of articulation joint 146. This alignment may allow for a continuous pathway for steering elements 206 or other control elements to extend from adapter 156 into articulation joint 146. The positioning of element grooves 202 on internal surface 204 of distal portion 156D may correspond to the arrangement of channels 164 within articulation joint 146, facilitating a smooth transition of internal components between the two structures.

Adapter 156 may be attached to the distal end of tubular member 119. Proximal portion 156P of adapter 156 may be configured to fit over the distal end of tubular member 119. The larger diameter of proximal portion 156P, as compared to distal portion 156D, may allow for a secure fit around the distal end of tubular member 119. The smaller diameter of distal portion 156D may provide a stop for tubular member 119, inhibiting tubular member 119 from being advanced beyond a distal end of proximal portion 156P. In some implementations, adapter 156 may be attached to tubular member 119 using an adhesive. The adhesive may be applied to the inner surface of proximal portion 156P, the outer surface of the distal end of tubular member 119, or both, before fitting the components together.

Alternatively, adapter 156 may be mechanically fastened to tubular member 119. For instance, proximal portion 156P may include one or more holes or slots through which fasteners such as screws or pins may be inserted to secure adapter 156 to tubular member 119. These fasteners may engage with corresponding features in tubular member 119. In other aspects, adapter 156 may be attached to tubular member 119 using a press-fit or interference fit. The inner diameter of proximal portion 156P may be slightly smaller than the outer diameter of the distal end of tubular member 119, allowing for a tight, friction-based connection when the components are pressed together.

Adapter 156 may also incorporate locking mechanism(s) to secure its attachment to tubular member 119. For example, proximal portion 156P may include internal locking tabs or a bayonet-style connection that engages with corresponding features of tubular member 119 when adapter 156 is twisted or pushed onto tubular member 119. In another example, adapter 156 may be fixed to tubular member 119 via a threaded fit. For example, an internal surface of proximal portion 156P may include threads, and an outer surface of the distal end of tubular member 119 may include receiving threads.

In some implementations, adapter 156 may be overmolded directly onto tubular member 119. This manufacturing process may involve positioning the distal end of tubular member 119 within a mold and injecting a moldable material around it to form adapter 156. The overmolding process may create a secure connection between adapter 156 and tubular member 119, potentially enhancing the structural integrity of the joint between these components. Overmolding adapter 156 directly onto tubular member 119 may eliminate the need for additional attachment mechanisms, such as adhesives or mechanical fasteners. This may simplify the manufacturing process and reduce the number of potential failure points in the connection between adapter 156 and tubular member 119.

The overmolding process may allow for the incorporation of various features directly into adapter 156 during its formation. For example, ribs 196, element grooves 202, and proximal locking tabs 198 may be formed as integral parts of adapter 156 during the overmolding process. This may result in a more robust and precisely formed adapter 156.

It will be apparent to those skilled in the art that various modifications and variations can be made to the disclosed device without departing from the scope of the disclosure. For example, the articulation joint described herein is designed as a single-component structure with living hinges connecting adjacent links. The single-component structure may provide advantages in manufacturing efficiency and durability. An adapter fixed to a distal end of a shaft may connect the articulation joint to the shaft. The adapter may include protrusions, locking tabs, and element grooves that align with the articulation joint's channels to provide a secure connection between the adapter and the articulation joint. Aspects of the adapter may facilitate the passage of steering wires and other internal components into and through the articulation joint. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1. A medical device comprising:
a handle;
a shaft extending from the handle;
an adapter fixed to a distal end of the shaft, the adapter having a proximal portion and a distal portion, wherein the distal portion includes a rib extending approximately parallel to a longitudinal axis of the shaft and a locking tab; and
an articulation joint having a proximal-most link, the proximal-most link including:
a proximal cavity configured to receive the distal portion of the adapter;
an opening on a radially outer surface of the proximal-most link, wherein the opening is configured to receive the locking tab of the adapter; and
a cutout configured to receive the rib of the adapter.

2. The medical device of claim 1, wherein the medical device further includes a steering wire that extends through the shaft, wherein the adapter further comprises a groove disposed on an internal surface of the distal portion, and wherein the steering wire is received in the groove.

3. The medical device of claim 2, wherein the groove is circumferentially aligned with the rib.

4. The medical device of claim 2, wherein the groove tapers to a point at a proximal end.

5. The medical device of claim 4, wherein a radial depth of the groove increases from the proximal end to a distal end of the groove.

6. The medical device of claim 1, wherein the rib is one of four ribs distributed circumferentially around the distal portion.

7. The medical device of claim 1, wherein the locking tab is disposed on a radially outer surface of the rib.

8. The medical device of claim 1, wherein the locking tab is configured to deflect during insertion of the distal portion of the adapter into the proximal cavity of the proximal-most link and return to an un-deflected position when aligned with the opening.

9. The medical device of claim 1, wherein the adapter is overmolded directly onto the distal end of the shaft.

10. The medical device of claim 1, wherein the adapter includes a ledge between the proximal portion and the distal portion, and wherein the distal end of the shaft terminates proximally of the ledge.

11. The medical device of claim 10, wherein a proximal-most edge of the proximal-most link abuts the ledge.

12. The medical device of claim 1, wherein the proximal-most link includes an internal wall, wherein the internal wall is distal of a proximal-most end of the proximal-most link, wherein the internal wall defines a distal-most end of the proximal cavity.

13. The medical device of claim 12, wherein the cutout is circumferentially aligned with a channel extending through the articulation joint.

14. The medical device of claim 1, wherein the opening is circumferentially aligned with the cutout.

15. The medical device of claim 1, wherein the locking tab is a first locking tab, wherein the articulation joint further comprises a distal-most link, and wherein the distal-most link includes a second locking tab extending radially inward relative to an internal surface of the distal-most link.

16. A medical device comprising:
a handle;
a shaft extending distally from the handle;
an articulation joint comprising a plurality of links, wherein the articulation joint includes a proximal-most link having a proximal cavity; and
an adapter connecting a distal end of the shaft to the articulation joint, the adapter comprising: a proximal portion coupled to the distal end of the shaft; and a distal portion received within the proximal cavity of the proximal-most link, wherein the distal portion includes a protrusion extending radially outward; and wherein the proximal-most link of the articulation joint includes an opening configured to receive the protrusion of the adapter, the opening and the protrusion cooperating to resist rotation between the adapter and the articulation joint.

17. The medical device of claim 16, wherein the distal portion of the adapter includes a rib extending radially outward from an outer surface of the distal portion, wherein the rib engages with an inner surface of the proximal cavity of the proximal-most link.

18. The medical device of claim 16, wherein the medical device further comprises a steering wire, wherein the adapter further comprises a groove disposed on an internal surface of the distal portion, and wherein the groove receives the steering wire.

19. The medical device of claim 18, wherein the groove tapers to a point at a proximal end of the groove, and wherein a radial depth of the groove increases from the proximal end to a distal end of the groove.

20. A medical device adapter comprising:
a proximal portion configured to couple to a distal end of a shaft; and
a distal portion configured to be received within a proximal cavity of an articulation joint, the distal portion including: a locking tab disposed on a radially outer surface of the distal portion; and a groove disposed on an internal surface of the distal portion, wherein the groove is configured to accommodate a steering wire; wherein the groove is circumferentially aligned with a rib; and wherein the groove tapers to a point at a proximal end of the groove, wherein a radial depth of the groove increases from the proximal end to a distal end of the groove.
